(19) United States
(12) Patent Application Publication
(10) Pub. No.: US 2024/0180469 A1
(43) Pub. Date: Jun. 6, 2024

(54) TEXTILE ELECTRODE CONNECTIONS

(71) Applicant: Propel, LLC, Pawtucket, RI (US)

(72) Inventors: Clare King, Providence, RI (US); Anjali Khemani, Providence, RI (US); Birgit Leitner, Providence, RI (US)

(21) Appl. No.: 18/436,210

(22) Filed: Feb. 8, 2024

Related U.S. Application Data
(63) Continuation-in-part of application No. 16/845,781, filed on Apr. 10, 2020, now Pat. No. 11,905,627.
(60) Provisional application No. 62/832,098, filed on Apr. 10, 2019.
(60) Provisional application No. 62/832,101, filed on Apr. 10, 2019.
(60) Provisional application No. 62/832,104, filed on Apr. 10, 2019.

(51) Int. Cl. A61B 5/27; A61B 5/00; A61B 5/256; A61B 5/265; B23K 26/352; D04B 1/10; D04B 21/06; D06C 7/02; D06M 10/00; D06M 11/83; D06M 15/55

(52) U.S. Cl. CPC A61B 5/27; A61B 5/256; A61B 5/265; A61B 5/6804; B23K 26/352; D04B 1/10; D04B 21/06; D06C 7/02; D06M 10/005; D06M 11/83; D06M 15/55; A61B 2562/125; D06M 2200/00; D10B 2401/063; D10B 2401/18; D10B 2403/0122; D10B 2509/00

(57) ABSTRACT

A knitted textile includes a textile electrode region, a conductive trace region that terminates in a knitted extension, conductive material, located at an intersection of an ablated area and the textile electrode region, configured to provide an electrical connection between the conductive trace region and the textile electrode region, sealing film, placed around the conductive material, configured to protect the conductive material and seal the conductive material from one or more textile layers that surround the electrical connection, and an outer sealing patch surrounding the textile electrode region and configured to provide a moisture barrier between the textile electrode region and the one or more surrounding textile layers. The conductive trace region includes one or more electrical conductors twisted with an insulator. The knitted extension is configured to overlay a portion of the textile electrode region and includes the ablated area where the insulator has been removed.

### CROSS-REFERENCE TO RELATED APPLICATIONS

This application is a Continuation-in-Part of pending U.S. Non-Provisional application Ser. No. 16/845,781, filed Apr. 10, 2020 and entitled SYSTEMS FOR MAINTAINING MOISTURE IN A TEXTILE ELECTRODE, which claims priority from U.S. Provisional Application Ser. No. 62/832,098 filed Apr. 10, 2019 and entitled GARMENTS WITH INTEGRATED ELECTRODES AND CONDUCTIVE TRACES, from U.S. Provisional Application Ser. No. 62/832,101 filed Apr. 10, 2019 and entitled SYSTEMS AND METHODS FOR MAINTAINING MOISTURE IN A TEXTILE ELECTRODE, and from U.S. Provisional Application Ser. No. 62/832,104 filed Apr. 10, 2019 and entitled HYBRID YARN FOR WEAVING CONDUCTIVE WIRES INTO FABRIC. The contents of all the above noted provisional patent applications and the parent non-provisional patent application are each hereby incorporated in their entireties by reference.

### GOVERNMENT RIGHTS

This invention was made with Government support under Funding Agreement No. N00189-17-C-Z023 awarded by the U.S. Navy. The Government has certain rights in the invention.

### FIELD

This disclosure relates to a method for assembling an electrical connection to a textile electrode worn against the skin.

### BACKGROUND

It is known to use textile electrodes for measuring physiological parameters of the human body. The use of textile electrodes, however, is constrained by their high impedance when their conducting material is dry. In addition, electrodes constructed from conductive threads woven or knitted together and placed against bare skin obtain relatively poor physiological signal quality (e.g., an electrocardiographic which is representative of the heart activity of a user) as compared to traditional electrodes which often use a highly conductive fluid or gel to place the electrode or conductive element in electrical contact with the user's skin. The gel or fluid reduces the impedance in contact with the electrode so that very small changes of electrical signals such as those measured by electroencephalography (EEG), electrocardiography (ECG) and electromyography (EMG) can be measured.

Prior art textile electrodes known by the inventors have attempted to improve their signal quality by ensuring the presence of moisture between the electrodes and the skin to allow ionic conduction between the two interfaces and thus, obtain a sufficient conductivity to detect signals generated by the human body. Typical system either provide a source of fluid to the electrode that maintains a moisture level between the electrode and the user's skin or rely on sweat generated by the user using physical activity to maintain a moisture level. The latter approach is highly dependent on the user's sweat output and level of physical activity, and severely limits the usefulness of the textile electrode. The former has to contend with the high level of fluid evaporation and absorption that can make the performance of the electrode unpredictable as the moisture level fluctuates depending on the user activity and environment.

In response, the prior art has taken one of two common approaches to maintain the moisture level in a textile electrode. The first adds a separate fluid reservoir and a system for moving fluid from the reservoir to the electrode. The second places a wetted material behind the electrode and separates the two by a semi-permeable membrane that allows moisture to flow from the wetted material to the electrode.

Both approaches, however, have serious drawbacks. Reservoir systems, for example, add a bulky fluid container that must be placed somewhere on the user, and can require an active transport mechanism to move fluid to the electrode. Systems with semi-permeable barriers are difficult to rewet, dry, and clean, which makes their wetted material prone to bacteria growth and breakdown.

### SUMMARY

The present disclosure relates to a system for providing an electrical connection to a textile electrode. Certain embodiments of the present disclosure provide for a knitted textile. The knitted textile includes a textile electrode region, a conductive trace region that terminates in a knitted extension, conductive material, located at an intersection of an ablated area and the textile electrode region, configured to provide an electrical connection between the conductive trace region and the textile electrode region, sealing film, placed around the conductive material, configured to protect the conductive material and seal the conductive material from one or more textile layers that surround the electrical connection, and an outer sealing patch surrounding the textile electrode region and configured to provide a moisture barrier between the textile electrode region and the one or more surrounding textile layers. The conductive trace region includes one or more electrical conductors twisted with an insulator. The knitted extension is configured to overlay a portion of the textile electrode region and includes the ablated area where the insulator has been removed.

In one embodiment, the textile electrode region and a portion of the conductive trace region that does not include the knitted extension are knitted in a common layer of the knitted textile.

In one embodiment, each knit stitch at the edge of the knitted extension is interlinked with a corresponding knit stitch of the textile electrode region except on one side of the knitted extension, wherein a pocket is formed between the knitted extension and the textile electrode region.

In one embodiment, the pocket is formed on an outside surface of the knitted textile.

In one embodiment, the conductive material is a silver paste compound.

In one embodiment, a laser creates the ablated area in the knitted extension.

In one embodiment, the knitted textile also includes a moisture reservoir, positioned above the sealing film on an outer side of the knitted textile and in contact with the textile electrode region, configured to retain moisture and enhance electrical transfer between the textile electrode region and skin of a user wearing the knitted textile and an outer seal patch, applied to the first side of the knitted textile and over the moisture reservoir, configured to surround the textile electrode region, limit evaporation of moisture from the moisture reservoir, and dampen electrical noise through the textile electrode region.

In one embodiment, insulators of the conductive trace region have a higher tensile strength than the one or more electrical conductors.

In one embodiment, the knitted textile includes a textile electrode, a conductive trace that terminates in a knitted extension, conductive epoxy, located at an intersection of the ablated area and the textile electrode and configured to provide an electrical connection between the conductive trace and the textile electrode, and adhesive film, applied to a first side of the knitted textile around and over an ablated area and the conductive epoxy and on a second side of the knitted textile opposite the adhesive film applied to the first side. The adhesive film is configured to provide a water-tight encapsulation of the electrical connection when cured. The knitted extension is configured to overlay a portion of the textile electrode and includes the ablated area where the insulator has been removed. The conductive trace includes one or more electrical conductors twisted with an insulator.

In one embodiment, a method for creating an electrical connection in a knitted textile includes intarsia knitting a first layer including a textile electrode and a conductive trace, the conductive trace including one or more conducting wires twisted with an insulator, the conductive trace terminating in a knitted extension forming a second layer and overlaying a portion of the textile electrode, ablating a portion of the knitted extension by removing the insulator from the ablated portion, applying a silver epoxy to the ablated portion, the silver epoxy providing an electrical connection between the conductive trace and the textile electrode, and applying an adhesive film to a first side of the knitted textile around and over the ablated area and the silver epoxy, and to a second side of the knitted textile opposite the adhesive film applied to the first side, the adhesive film providing a water-tight encapsulation of the electrical connection.

Other implementations, features, and advantages of the subject matter included herein will be apparent from the description and drawings, and from the claims.

### BRIEF DESCRIPTION OF THE DRAWINGS

This disclosure will be more fully understood from the following detailed description taken in conjunction with the accompanying drawings, in which:

FIG. 1A is a schematic illustration of a single-layer textile formed as a wearable garment with integrated textile electrodes and conductive traces connecting the electrodes to a controller unit configured in accordance with illustrative embodiments.

FIG. 1B is an illustrative embodiment of the textile of FIG. 1A on a user.

FIG. 2A is a photograph of an illustrative embodiments of a hybrid yarn.

FIG. 2B is a schematic illustration example twist patterns of a conductive wire around a nonconductive yarn.

FIG. 3 is a rendering of an embodiment of a knitted textile having a textile electrode and a conductive trace region with a loose loop of hybrid yarn from the conductive trace extending across the textile electrode.

FIG. 4 is a schematic illustration of a single layer of a continuous textile section knitted having a conductive trace region passing through an inert region and electrically connected to an electrode region of the textile section.

FIG. 5 is a schematic illustration of a reservoir system for maintaining moisture in a textile electrode in accordance with illustrative embodiments of the invention.

FIG. 6 is schematic illustration of a reservoir system for maintaining moisture in a textile electrode showing the connection between the textile electrode and a conductive wire of a hybrid yarn in accordance with various embodiments.

FIG. 7 is a schematic illustration of a reservoir system of FIG. 6 showing the connection between the textile electrode and the conductive wire in more detail.

FIG. 8 is a photograph of a knitted textile embodiment having a textile electrode and a conductive trace extending into the textile electrode, showing the location of an electrical connection and a film seal.

FIG. 9 is a schematic drawing of one embodiment of a reservoir system for maintaining moisture in a textile electrode showing the connection between the textile electrode and a conductive wire of a hybrid yarn.

FIG. 10 is a photograph of an illustrative textile with a textile electrode connected to a conductive trace and covered by a piece of wool.

FIG. 11 is a schematic illustration of a single layer of a continuous textile section knitted using the intarsia technique and having a conductive trace region passing through an inert region and across a face of an electrode region.

FIGS. 12A-12G are photographs of an embodiment of the steps for coupling a conductive trace region of a knitted textile to an integrated electrode region of the knitted textile by ablating a portion of the conductive trace region that extends across the integrated electrode, applying a conductive material to the ablated portion, and sealing the resulting electrical connection.

FIG. 13 shows a process of creating an electrical connection between the textile electrode and a conductive trace of the knitted textile in accordance with illustrative embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Example Textiles with Integrated Conductive Traces

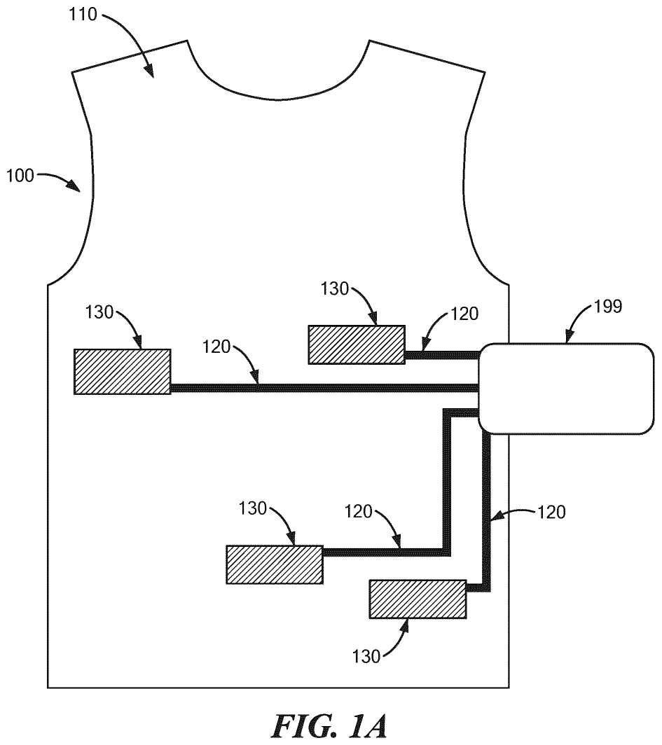

FIG. 1A is a schematic illustration of a textile formed as a wearable garment with integrated electrodes and conductive traces connecting the electrodes to a controller unit configured in accordance with illustrative embodiments. Specifically, FIG. 1A schematically shows a textile garment 100 with integrated textile electrodes 130 and conductive traces 120 connecting the textile electrodes 130 to an electrical device 199. The garment 100 may be constructed as a single textile layer to be worn directly against the skin. The garment 100 is knitted from a regular electrically inert material 110 (e.g., an insulator material, such as cotton, wool, or polyester) with the textile electrodes 130 knitted directly into the garment 100, without adding additional textile layers at the location of the textile electrodes 130. Being a single layer, a thickness at any point on the garment 100 includes either electrically inert material 110, a conductive trace 120, or a textile electrode 130, but none of the materials 110, 120, 130 in combination. The conductive traces 120 are knitted with a hybrid yarn, discussed in more detail below, that is constructed from a strong and inelastic nonconductive yarn twisted with one or more conductive wires, with the conductive wires being coated with an insulating material in one embodiment. The hybrid yarn enables the conductive traces 120 to transmit power or electrical signals through the conductive wires without interference due to the insulating coating on the conductive wires. The textile electrodes 130 have an inner surface that is therefore positioned against the user's skin when the garment 100 is worn. The textile electrodes 130 are knitted from a conductive yarn, such as a silver coated polyester, that enables the textile electrodes 130 to conduct electrical signals across the textile electrode 130. The textile electrodes 130 are connected to the electrical device 199 via conductive traces 120 that are also knitted directly into the garment 100 without adding additional layers to the garment. In some embodiments, the garment 100 defines a single knitted textile layer across the inert material 110, the textile electrodes 130, and the conductive traces 120. In some embodiments, the textile electrodes 130 are knitted as electrical connection regions for a sensor or electronic device affixed to the garment 100.

The textile electrodes 130 may be arranged to, for example, pick up or sense electrical signals from the user's body, such as those related to heart rate and heart function (e.g., the signals for use in forming an electrocardiogram or EKG). In some embodiments, the garment 100 includes four textile electrodes 130, positioned with respect to the user's body to provide a high-quality EKG signal. The conductive traces 120 may connect the textile electrodes 130 to the electrical device 199 via the conductive wires integrated into the hybrid yarn from which the conductive traces 120 are knitted. The conductive wire of the hybrid yarn may be coated with an insulating polymer, which is able to be removed at the points of contact with the textile electrodes 130 and the electrical device 199.

In some embodiments, the hybrid yarn may be constructed from a highly inelastic material, such as meta-aramid or para-aramid (e.g., Kevlar® or Twaron®) or a material with similar material properties to protect the integrated conductive wires from damage or being severed during the knitting process and being damaged or severed during normal wear of the garment 100, such as Ultra High Molecular Weight Polyethene (UHMWPE), Polybenzimidazole (PBI), Polyphenylene Benzobisoxazole (PBO), High Strength Polyester, Liquid-Crystal Polymer (LCP), or spider silk. In some embodiments, the hybrid yarn is made with a fire retardant and self-extinguishing material, such as para-aramid or material with similar properties according to the ASTM D6413/D6413M Standard Vertical Test Method for Flame Resistance of Textiles to enable the insulating layer and nonconductive yarn to be removed using ablation. In one embodiment, the hybrid yarn may be V8 by Propel LLC, which includes two conductive copper-clad stainless steel wires twisted with a Kevlar non-conductive strand at 5 to 12 twists per inch (TPI). The conductive wire may be, for example copper wire or copper-clad stainless-steel wire. Additionally, the textile electrodes 130 may be knitted or otherwise constructed with a conductive wire, such as silver or copper wire or a nonconductive yarn (e.g., nylon, polyester, cotton, or wool) coated with a conductive material such as silver or copper. In one embodiment, the textile electrodes 130 are knitted from a silver-plated yarn, such as AGPoss from Mitsufuji Corporation. In some embodiments, the electrically inert material 110, textile electrodes 130, and conductive traces 120 are knitted together into a single-layer garment 100 without seams.

FIG. 1B is an illustrative embodiment of the textile of garment 100 FIG. 1A on a user. FIG. 1B shows patches 130′ over the textile electrodes 130 that are arranged to maintain a moisture level in the textile electrode 130. These patches 130′ can also be used to impart stability to the textile electrode on body when the garment is worn and to reduce electrical static noise from the outer surface of the textile electrode 130.

### Example of a Hybrid Conductive Yarn

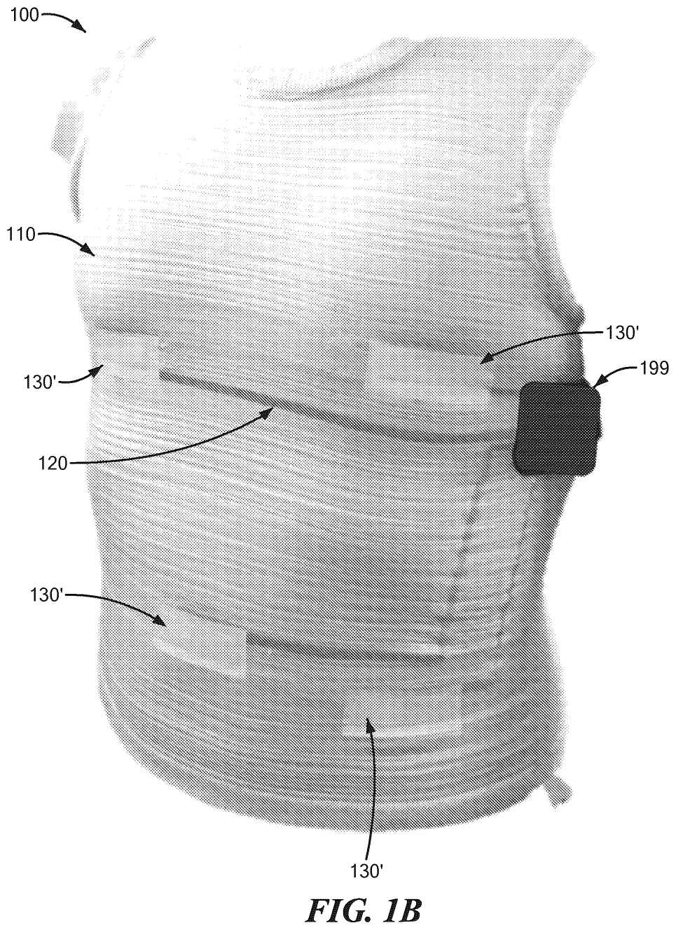

FIG. 2A is a photograph of a strand of a hybrid yarn 200 configured in accordance with illustrative embodiments. To show its relative size, the hybrid yarn 200 is compared with a US penny coin and a strand of human hair. Preferably, the hybrid yarn 200 is made from a nonconductive yarn 210 or insulator and one or more conductive wires 220 twisted together. In some instances, the nonconductive yarn 210 may have minimal elasticity and high strength, and may be made from, for example, a meta-aramid or para-aramid material. The nonconductive yarn 210 also may be made from filament or staple fibers. The conductive wire 220 may be insulated with, for example, a polyurethane or Teflon coating. In some instances, the hybrid yarn 200 may be bonded with a coating (e.g., Nylon) for a softer feel while maintaining the integrity of the hybrid yarn 200.

In one example, the hybrid yarn 200 may include two stands of copper-clad stainless steel or copper with between 5 to 12 twists per inch around a Kevlar strand. The 5 to 12 twists per inch construction is for a strand of Kevlar and a 50 micron conductive wire (e.g., 43 micron thick metal and a 3-4 micron thick coating of polyurethane) that when twisted together suitable to knit a textile at 15 gauge. The hybrid yarn in FIG. 2 may be made from two copper clad stainless-steel wires 220 twisted with a Kevlar yarn 122 at 9 twists per inch. In some instances, other nonconductive yarns 210 may be used, such as Vectran® or Twaron®, which are also a high strength yarns with low elasticity.

Nonconductive yarns 210 made with para aramid or similar materials have many advantages, such as being strong but relatively light. The specific tensile strength (stretching or pulling strength) of both Kevlar 29 and Kevlar 49 is over eight times greater than that of steel wire. Unlike most plastics, it does not melt: it is reasonably good at withstanding temperatures and decomposes only at about 450° C. (850° F.). Accordingly, the hybrid yarn 200 may be laser ablated, burned, or exposed to chemicals to remove the nonconductive yarn 210 and the coating on the conductive wire 220.

FIG. 2B is a schematic illustration of an example twist pattern of a hybrid yarn 200 having a conductive wire 220 around a nonconductive yarn 210. In order to knit the conductive traces 120 into a single layer using a flatbed or 3D knitting machine, the nonconductive yarn 210 must protect conductive wire 220 from being broken by the stresses put on the hybrid yarn 200 by the flatbed knitting machine. According, a hybrid yarn 200 was developed that is suitable for flatbed knitting. The hybrid yarn 200 is constructed from the nonconductive yarn 210 being twisted with the conductive wire 220, where the nonconductive yarn 210 is a strong and inelastic yarn that, when exposed to the tensile forces of the flatbed knitting machine, exhibits an elongation of a sufficiently small percentage to prevent breakage of the conductive wire 220. For example, the nonconductive yarn 210 may have a tensile strength greater than that of the conductive wire 220 as well as an elongation break percentage less than 5 or less than about 4.2. In other embodiments, the nonconductive yarn 210 may have a Young's modulus of 60 or greater. In practice, because the nonconductive yarn 210 and conductive wire 220 are twisted together and the nonconductive yarn 210 comprises a majority fraction of an overall cross-section of the hybrid yarn 200, the material of nonconductive yarn 210 need not simply be less elastic than the metal of conductive wire 220 because, as the hybrid yarn 200 is exposed to tensile forces, the hybrid yarn 200 acts as a single structure and the relative elasticity of the much larger nonconductive yarn 210 section is less than the relative elasticity of the much thinner conductive wire 220 as the hybrid yarn 200 undergoes tension. Accordingly, suitable embodiments of hybrid yarn 200 may be constructed from very strong and inelastic fibers, such as meta-aramids and para-aramids, that are both thin and flexible enough to be knitted on a flatbed machine, but also strong and inelastic enough at those thin diameters to be twisted with a substantially thinner metal wire (e.g., a conductive wire 220 thin enough to maintain the thin and flexible properties of the overall hybrid yarn 200 that enable it to be both machine knittable and not affect the worn feeling of a garment) and prevent the substantially thinner metal wire from breaking.

### Examples of Connecting a Hybrid Conductive Yarn to a Textile Electrode

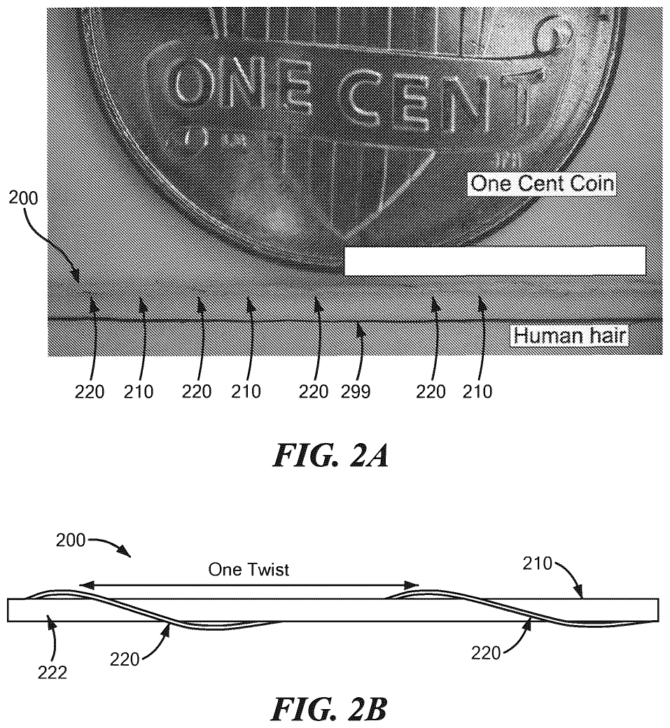

FIG. 3 is a photograph of an embodiment of a knitted textile having an integrated electrode region 130 or textile electrode 130 and a conductive trace region 120 or conductive trace 120 with a loose loop 123 of hybrid yarn 200 from the conductive trace region 120 extending across the face of the textile electrode region 130. The loop 123 can be cut into a tail to facilitate connection between the textile electrode region 130 and the conductive trace region 120 of which the loop or tail is an extension of the same hybrid yarn 200. The loose loop 123 may be used to electrically connect the conductive trace region 120 to the textile electrode region 130 by removing the insulating layer (and, in some embodiments, the nonconductive yarn) from the loop 123 and connecting the now-bare conductive wire 220 of the loop 123 to the conductive yarn 131 of the textile electrode region 130. Leaving this loop 123 loose allows the loop 123 to be ablated, exposing the bare conductive wire 220, without destroying the textile 100, 120, 130. In some embodiments, the loose loop 123 may beneficially increase the surface area of the conductive wire 220 that is able to be connected to the textile electrode 130, as well as providing a free strand to remove the insulating coating and nonconductive yarn more easily.

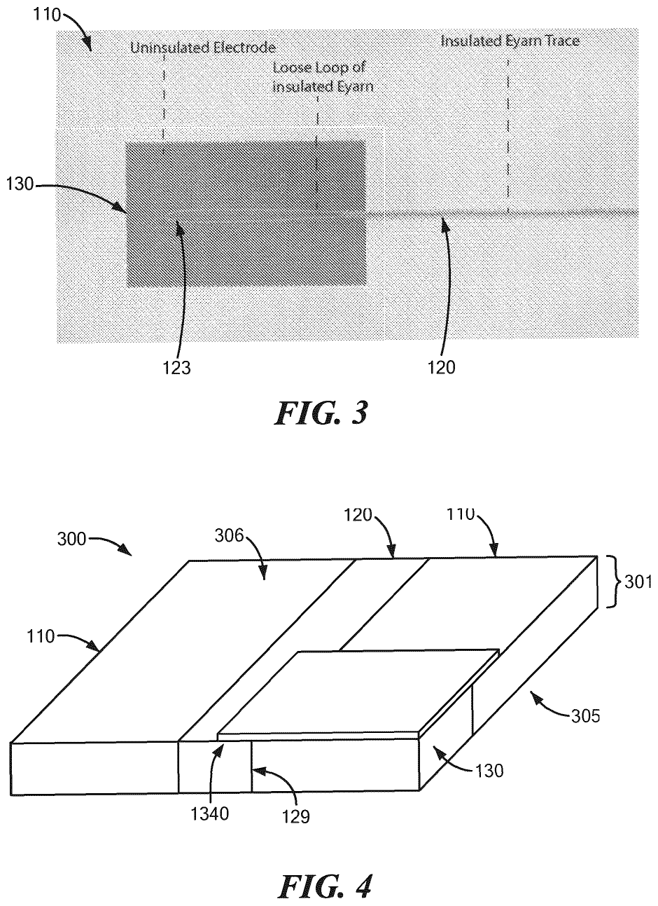

FIG. 4 is a schematic illustration of a single layer 301 of a continuous textile section 300 knitted to have a conductive trace region 120 passing through an inert region 110, with the conductive trace region 120 being electrically connected to a textile electrode region 130 of the textile section 300 at an interface 129 between the two regions. The single layer 301 defines a bottom side 305 and a top side 306 opposite the bottom side, with each region 110, 120, 130 extending between the top side 306 and the bottom side 305. Additionally, FIG. 4 shows a seal or patch 1340 positioned on the top side 306 of the textile electrode region 130.

### Example Moisture Retaining Systems

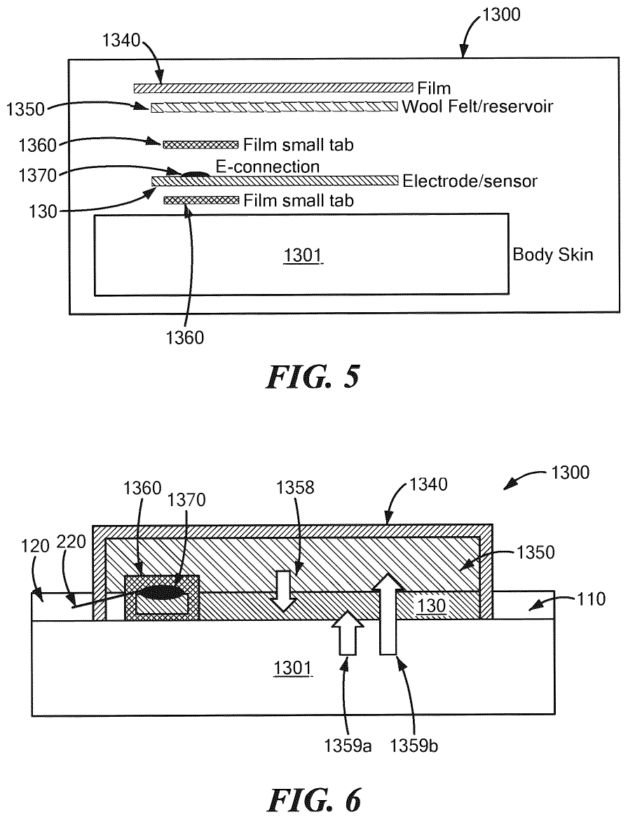

FIG. 5 is a schematic of a reservoir system 1300 for maintaining moisture in a textile electrode. The electrodes/sensors preferably are stabilized for electrical reasons. Among other benefits, stabilization of the electrode reduces noise, thus providing better data from the textile electrode region 130. Adding an outer film layer overcomes a constraint of a data gathering textile electrode—keeping it damp.

Specifically, data is more effectively captured when the textile electrode 130 is stable and damp. As such, illustrative embodiments add an outer film layer 1340 around the textile electrode region 130. For example, the added layer 1340 may include a thermoplastic adhesive cover film (or thermoplastic textile laminate) that mitigates evaporation of moisture from the region of the textile electrode region 130 through the garment 100. Moreover, adding an additional layer of fabric 1350, between the textile electrode region 130 and the film 1340 improves sweat absorption. Multiple tests were conducted with a variety of different materials used as the hydrophilic layer 1350, such as non-woven wool batting, dense polyester knit (brand name Axe suede) and superhydrophobic fiber and superhydrophobic yarn (as produced by Technical Absorbents, Grimsby, UK). Framis ‘Portofino’ laminate (polyester jersey+TPU adhesive) and Framis ‘Heavy Dream’ (TPU Cover-Film) was used as a stabilization ‘patch’. Here it was discovered that hydrophobic/hydrophilic materials, such as natural wool, are superior when used as the reservoir material. Natural wool absorbs salt water well and does not readily evaporate. Natural wool is also naturally fire resistant and has anti-microbial properties that are consistent with its intended use in this embodiment next to the skin of a user. Further, natural wool washes and dries without deterioration. Other hydrophobic materials, such as those tested, can also be used to form the reservoir 1350 but wool has the best characteristics for performance in the garment 100. The wool may be any form including loose fiber, or layers of knitted or woven wool, or felted wool, or non-woven wool batting. While some embodiments are 100% wool, wool blended with other fibers at no less than 70% wool/30% other fibers may also be used.

FIG. 5 shows a cross-section of a textile electrode region 130 of a garment positioned in-use, against a user's base skin 1301, with a reservoir system 1300 for retaining moisture in the textile electrode region 130. The reservoir system 1300 includes an outer film layer 1340 above a reservoir material 1350, which is itself directly above the textile electrode region 130. The electrode 130 may include an electrical contact 1370 that is separately sealed by an inner film 1360 from the rest of the textile electrode region 130, reservoir material 1350, and the user's skin 1301.

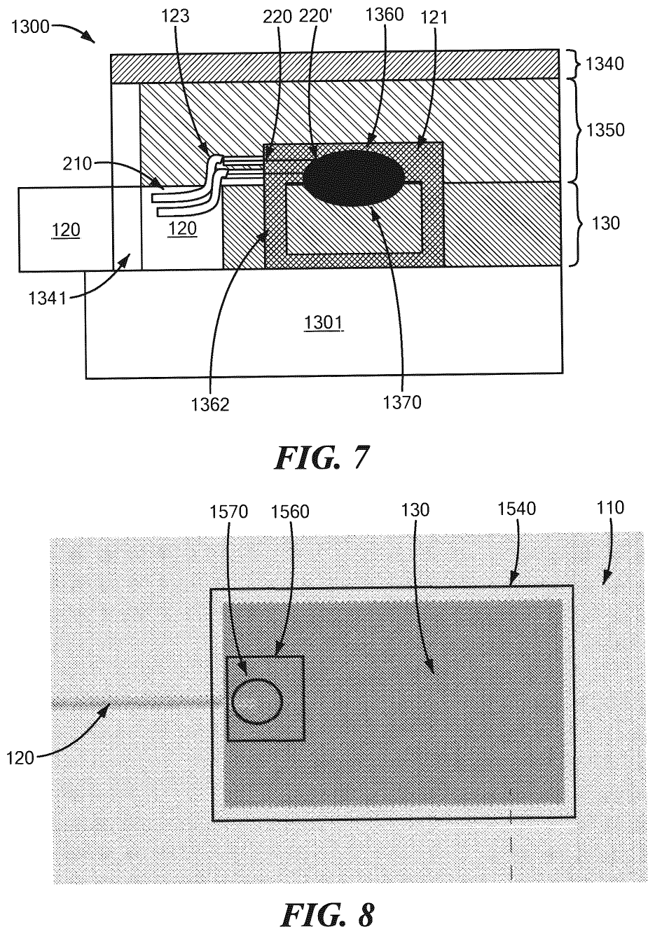

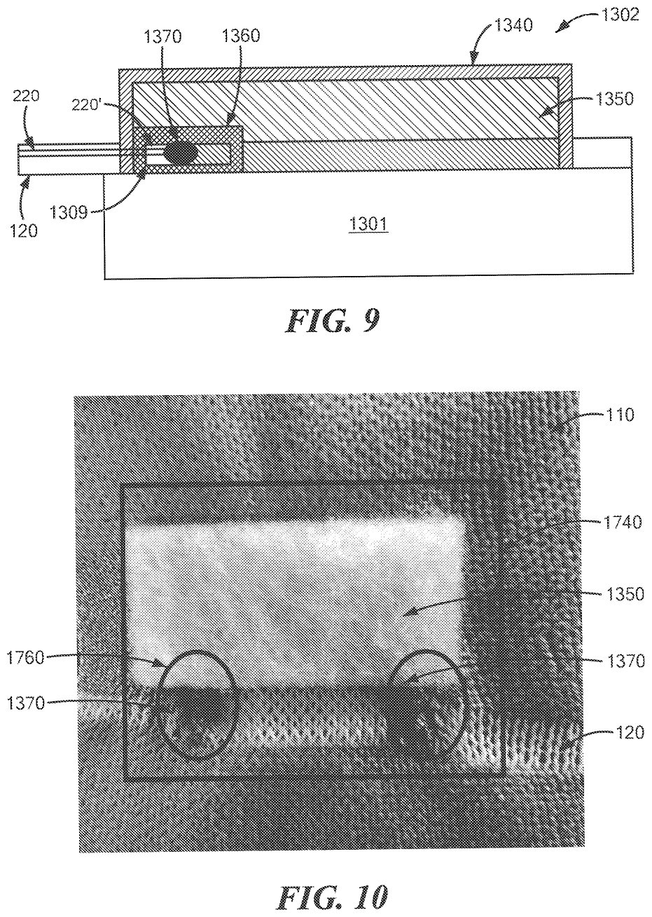

FIGS. 6 and 7 are schematic drawings of the reservoir system 1300, which maintains moisture in a textile electrode region 130. These figures also show the connection between the textile electrode region 130 and a conductive wire 220 of a conducive trace 120 made from a hybrid yarn. FIG. 6 shows the outer film layer 1340, surrounding the reservoir material 1350 and the textile electrode region 130, extending through the garment 100 (shown here as through the electrically inert material 110 on one side and the conductive trace 120 on the other) to the inner side of the garment abutted against the user's skin 1301. In this manner, the outer layer 1340 encapsulates the textile electrode region 130 and the reservoir material 1350 with a waterproof barrier against the user's skin 1301. Inside the outer layer 1340, water vapor from the reservoir material 1350 may flow into the textile electrode region 130 (shown as arrow 1358), and water vapor from the user's skin 1301 is able to flow into the electrode 130 (shown as arrow 1359a) and into the reservoir material 1350 (shown as arrow 1359b). FIG. 6 also shows that a conductive wire 220 from the conductive trace 120 extends though the outer film 1340 and is connected to the conductive material of the textile electrode region 130 with an electrical connection 1370. In some instances, and as shown, the electrical connection 1370 is encapsulated by an inner film 1360 that prevents moisture from the textile electrode region 130, reservoir material 1350, or the user's skin 1301 from reaching the electrical connection 1370.

FIG. 7 is a detail view of the connection between the conductive trace 120 and the textile electrode region 130. FIG. 7 shows that a loop 123 of hybrid yarn, including a coated conductive wire 220 and a nonconductive yarn 210 extending from the conductive trace 120, though the outer film 1340. FIG. 7 also shows uncoated portion 220′ of the conductive wire 220 extending through the inner film 1360 to the electrical connection 1370. In FIG. 7, a portion 1362 of the inner film 1360 may extend through the textile electrode region 130 to completely seal the electrical contact 1370 from moisture while still allowing the conductive material of the textile electrode region 130 to pass though the portion 1362 to maintain the electrical connection between the electrical contact 1370 and the rest of the textile electrode region 130.

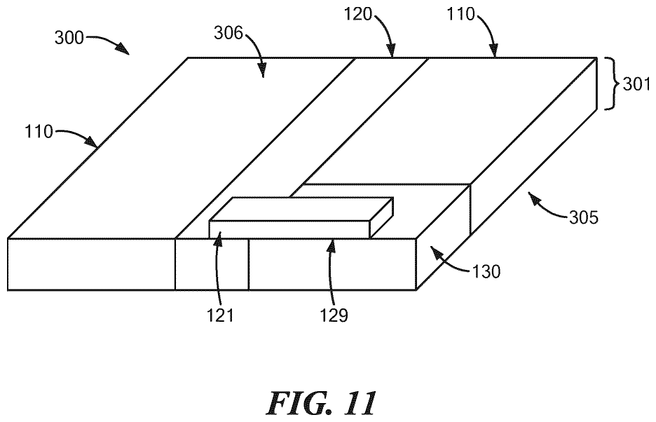

FIG. 8 is a graphic rendering of a knitted textile embodiment having a textile electrode 130 and a conductive trace 120 extending into the textile electrode 130, showing the location of an electrical connection and a film seal. In FIG. 8, a section of the knitted textile garment 100 may have an integrated textile electrode region 130 and a conductive trace 120 extending into the textile electrode region 130 after the knitting steps are completed. In operation, once the knitted textile, including the conductive trace 120 and integrated textile electrode region 130 is knitted, the conductive wires 220 of the conductive trace 120 can be connected to the conductive material of the electrode 130 by, for example, an ablation process, whereby a small section of an insulating portion of the textile where the conductive trace 120 meets the textile electrode region 130 is ablated away, leaving only the uncoated conductive wires 220′ behind. With the uncoated conductive wires 220′ exposed, an electrical contact 1370 (e.g., a conductive material, such as silver epoxy) may be added at the location of the exposed wires 220′ to electrically connect the exposed wires 220′ to the surrounding conductive material of the textile electrode region 130.

FIG. 8 shows the location of this electrical contact 1370 as a boxed region 1570. With the electrical contact 1570 created, an inner sealing layer or film 1560 may be placed around the electrical contact 1570 and though the surrounding textile 120, 130 such that the material of the inner film 1560 becomes integrated into the fibers of the surrounding textile 120, 130 to form a sealed moisture barrier around the electrical contact 1570, without interrupting the fibers of the textile electrode region 130, such that the electrical contact 1370 remains in electrical contact with the textile electrode region 130. With the inner sealing layer 1560 in place, the reservoir material 1350 may be placed above the electrode 130, and the outer sealing layer 1540 may seal the reservoir material 1350 and the textile electrode region 130 such that only the inner surface of the electrode 130 (i.e., the skin-facing surface) is exposed inside a perimeter of the sealing layer 1540 extending into the fibers of textile around the textile electrode region 130, such that the moisture retained by the reservoir 1350 cannot escape through the garment. FIG. 8 indicates the location 1570 where the electrical connection 1370 may be placed, the location 1560 where the inner layer 1360 can be placed, and a location 1540 where an outer layer 1340 may be integrated with the material of the garment 100 to surround the textile electrode region 130 and reservoir material 1350 after the reservoir material 1350 is placed on the textile electrode region 130.

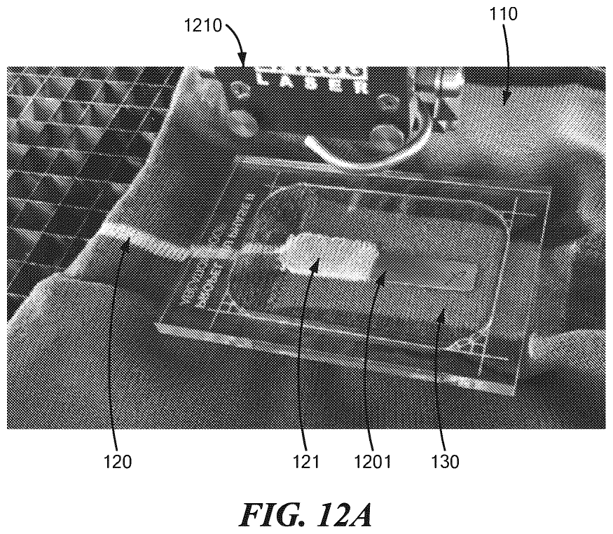

FIG. 9 is a schematic drawing of one embodiment of a reservoir system 1302 for maintaining moisture in a textile electrode 130, showing the connection between the textile electrode 130 and a conductive wire of a hybrid yarn. In FIG. 9, a schematic of an alternate configuration of the reservoir system 1302 for maintaining moisture in a textile electrode region 130 is shown. Among other things, FIG. 9 shows the connection between the textile electrode region 130 and a conductive wire 220 of a hybrid yarn 200 in a conductive trace 120 in the garment 100. The conductive trace 120 may be electrically connected to the textile electrode region 130 where the two knitted materials meet. For example, an ablated region 1309 of a loop of the conductive trace 120 may expose the uncoated conductive wire 220′. The ablated loop is then bundled up and attached to the structure of the knit textile electrode region 130. Here, an electrical contact 1370 may be placed to improve the connection between the uncoated wire 220′ and the textile electrode region 130. In some instances, the electrical contact 1370 may include a conductive plate or a bead of conductive metal, such as solder or a conductive adhesive or an uninsulated conductive yarn.

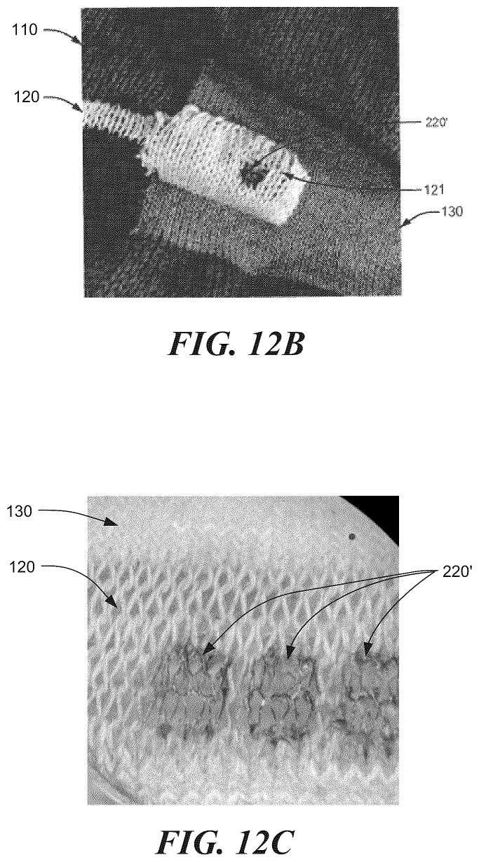

FIG. 10 is a photograph of an illustrative textile with a textile electrode connected to a conductive trace and covered by a piece of wool. FIG. 10 shows a section of a garment 100 with an integrated textile electrode region 130 connected to a conductive trace 120 and covered by a piece of reservoir material 1350 (shown here as a piece of felted natural wool). FIG. 10 shows the region around the textile electrode region 130 and reservoir material 1350, as well as a section of the conductive trace 120, to be sealed by the outer film 1340. FIG. 10 also shows the region 1760 around the two electrical contacts 1370 to be sealed by an inner film 1360.

### Examples of Assembling a Moisture Retaining System

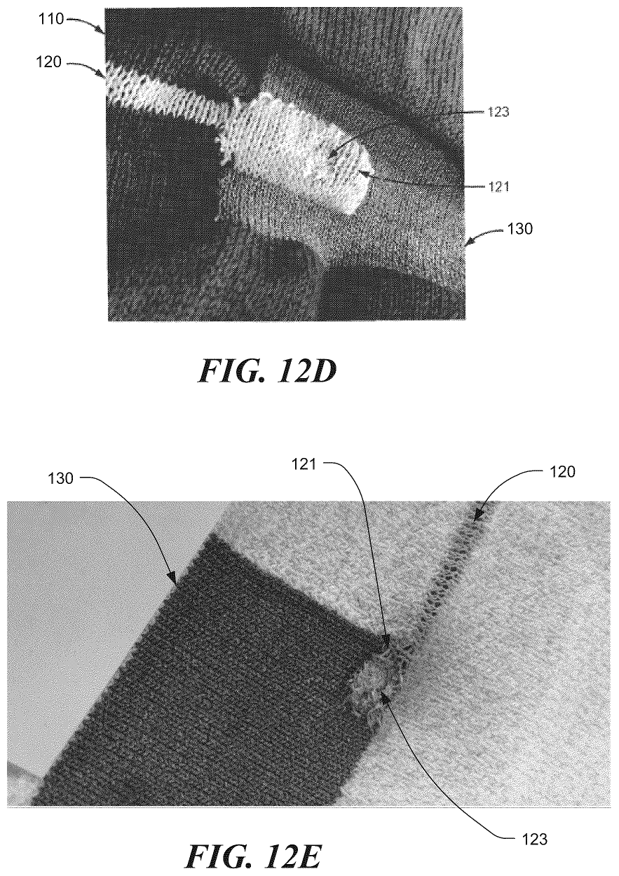

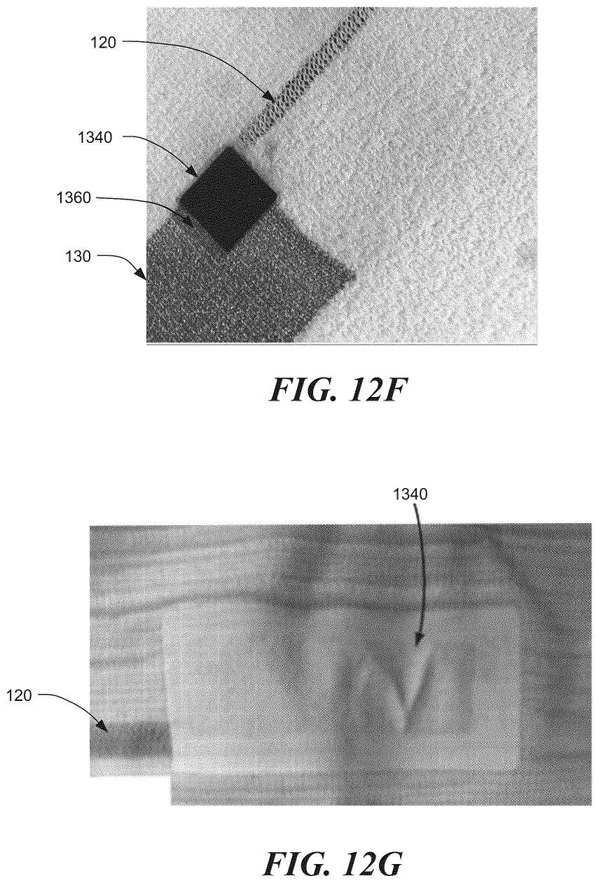

FIG. 11 is a schematic illustration of a single layer of a continuous textile section 301 knitted using the intarsia technique and having a conductive trace region 120 passing through an inert region 110 and across a face of an electrode region 130. The conductive trace region 120 includes a knitted extension 121 that is knitted out of the single layer of the continuous textile section to form a second layer above the textile electrode region 130. This knitted extensions 121 of the conductive trace region 120 can be electrically connected with the textile electrode region 130 as discussed in FIGS. 12A-12G.

FIGS. 12A-12G are photographs of an embodiment of the steps for coupling a conductive trace region 120 of a knitted textile to an integrated textile electrode region 130 of the knitted textile by ablating a knitted extension 121 of the conductive trace region 120 that extends across the integrated electrode.

In FIG. 12A, the knitted extension 121 is prepared for an ablation operation that removes non-conductive yarn 210 from a portion of the knitted extension 121. This is required prior to bonding the conductive wires 220′ of the conductive trace 120 to the textile electrode 130. First, edges of the knitted extension 121 may be interlinked with the underlying textile electrode 130, except on one side of the knitted extension 121. This forms a pocket between the knitted extension 121 and the textile electrode 130. Second, a protective structure 1201 may be inserted in the pocket. The protective structure 1201 serves as a heat shield to protect the textile electrode 130 while the non-conductive yarn 210 is being ablated from the knitted extension 121. The protective structure 1201 may be a thin metal plate or other form of heat shield material suitable for use in laser ablating operations. Interlinking each knitted stitch at the knitted extension 121 edges with corresponding knitted stitches of the textile electrode 130 also beneficially positions the knitted extension 121 in a fixed location relative to the textile electrode 130 to facilitate precise ablation. Third, the textile section 301 of FIG. 11 is positioned below a laser ablation tool with the protective structure 1201 disposed between the knitted extension 121 and the textile electrode region 130 to allow a portion of the knitted extension 121 to be ablated without damaging the underlying textile electrode region 130. Fourth, the laser ablation tool is activated to remove the non-conductive yarn 210 from the ablated area of the knitted extension 121. Fifth and finally, the protective structure 1201 is removed from the pocket.

In another embodiment, a portion of the knitted extension 121 may be treated with one or more chemicals to remove or ablate the non-conductive yarn 210. The choice of chemical(s) to use may depend on the composition of the non-conductive yarn 210 (e.g., a meta-aramid or para-aramid material, filament or staple fibers, etc.) and should be chosen appropriately.

In FIG. 12B, the bare conductive wire 220′ is now exposed in the portion of the knitted extension 121 that was ablated. For example, a laser such as an Epilog Fusion 75 Watt laser at 50% power and 25% speed may be used as the ablation tool, performing rastor engraving. As a result of the ablation operation, various forms of ash or chemical debris may be present in the ablated portion of the knitted extension 121. A suitable cleaning agent (e.g., acetone) should be applied to the ablated portion to remove any debris that may be present. For example, a soft-bristle toothbrush may be used to apply the acetone to the ablated area and X and Y-direction brushing may remove debris.

In FIG. 12C, a close-up view of three ablated sections of a conductive trace 120 is shown. Conductive wires 220′ are visible in each, formed as interlocking loops from intarsia knitting. Although the ablated portion of the knitted extension 121 may include three separate ablated sections, as shown, a single ablated portion of the knitted extension 121 may be preferable to minimize the size and weight of the electrical connection between the knitted extension 121 and the textile electrode 130.

In FIG. 12D, a conductive adhesive or similar conductive material 123 has been placed in and around the ablated portion of the knitted extension 121 with the bare conductive wire 220′ to electrically connect the conductive wire 220 of the conductive trace 120 with the textile electrode 130. The conductive adhesive 123 should also be placed on an opposite side of the knitted textile on the textile electrode 130, with a location or position coincident with the ablated portion on the other side of the textile electrode 130. In a preferred embodiment, the conductive adhesive is a silver epoxy in paste form.

In FIG. 12E, the conductive adhesive 123 has been applied to the ablated portion of the knitted extension 121. Preferably, the conductive adhesive 123 is pushed downward through the conductive wires 220′ of the knitted extension 121 to make direct contact at several points with the textile electrode 130. To facilitate a thorough electrical connection, a thin flexible plastic layer may be overlaid to the conductive adhesive 123 and a weight placed on the thin plastic layer (e.g., ½″ steel nut) until the conductive adhesive 123 has fully cured. The thin plastic sheet may also be placed on the other side of the textile electrode 130, where the conductive adhesive 123 was placed. Following curing (e.g., 12 hours or as recommended by the manufacturer) the weight and thin plastic later may be removed. In one embodiment, the conductive adhesive 123 may be a silver conductive epoxy adhesive such as MG chemicals #8331. The conductive adhesive 123 beneficially provides a large advantage over conventional electrical connection processes, such as soldering, by providing a more flexible connection that is better able with withstand machine washing and drying processes.

In FIG. 12F, a sealing film 1360, such as Sealon 3099, has been placed around the conductive material 123 and knitted extension 121 to fully encapsulate it and seal it from moisture that may be present in the surrounding textile layers 120, 130. The sealing film 1360 has an adhesive on one side and is a water barrier to water in a liquid or vapor state. In one embodiment, the sealing film 1360 may be cut to approximately 0.5″×0.5″ square and placed on both sides of the knitted textile 100. In a preferred embodiment, the sealing film 1360 placed over the knitted extension 121 is the same size and coincidently located with the sealing film 1360 placed on the opposite side of the textile electrode 130. Once placed, a hot press heats the sealing film 1360 on both sides of the textile electrode 130. This causes the top and bottom sealing film 1360 to melt through the porous knitted extension 121 and textile electrode 130 and combine with melted sealing film 1360 on the opposite side. This beneficially forms an encapsulated electrical connection. Following heating the sealing film 1360 for a predetermined heating time (e.g., 5 seconds for hot press), a cold press is applied to the sealing film 1360 to cure the sealing film 1360. In one embodiment, the sealing film 1360 may be cooled for 10 seconds by the cold press to complete the sealing operation for the electrical connection. Following curing of the sealing film 1360, an outer sealing patch 1340 may be affixed, as described below.

In FIG. 12G, an outer sealing patch 1340 is placed completely around the entire textile electrode region 130 on the outside surface of the knitted textile to create a moisture barrier between the textile electrode region 130 and the rest of the knitted textile. To be effective, all edges of the outer sealing patch 1340 must be outside the outside edges of the textile electrode 130 to provide a moisture seal around the back and sides of the textile electrode 130. In some embodiments, a reservoir material 1350 is also placed between the textile electrode 130 and the outer sealing patch 1340 to retain moisture in the textile electrode 130 and maintain the sensing performance of the textile electrode 130 as the knitted textile remains against the skin 1301. The outer sealing patch 1340 has an adhesive on one side and is a water barrier to water in a liquid or vapor state. In one embodiment, all loose yarn ends at the inside of the garment 100 should be clipped close to the garment 100 and removed.

The outer sealing patch 1340 is only placed on the outside of the knitted textile because the textile electrode 130 must be in direct contact with a user's skin 1301. Once the outer sealing patch 1340 is placed around the textile electrode 130, the outer sealing patch 1340 is cured by a similar process as the sealing film 1360. Hot press and cold press temperatures and durations may be similar to the sealing film 1360, or different, and manufacturer recommendations should be followed. When the outer sealing patch 1340 is heated, edges of the patch may melt through the thickness of the textile electrode such that the sealing patch 1340 extends to a user's skin 1301 when wearing the garment 100. In one embodiment, the outer sealing patch 1340 may be SKNTEX by Propel LLC or DREAM by Framis. Following heating the outer sealing patch 1340 for a predetermined heating time (e.g., 18 seconds for hot press), a cold press is applied to the outer sealing patch 1340 to cure the outer sealing patch 1340. In one embodiment, the outer sealing patch 1340 may be cooled for 15 seconds by the cold press to complete the sealing operation for the electrical connection. Following this step, all electrical connections should be verified for integrity and consistency.

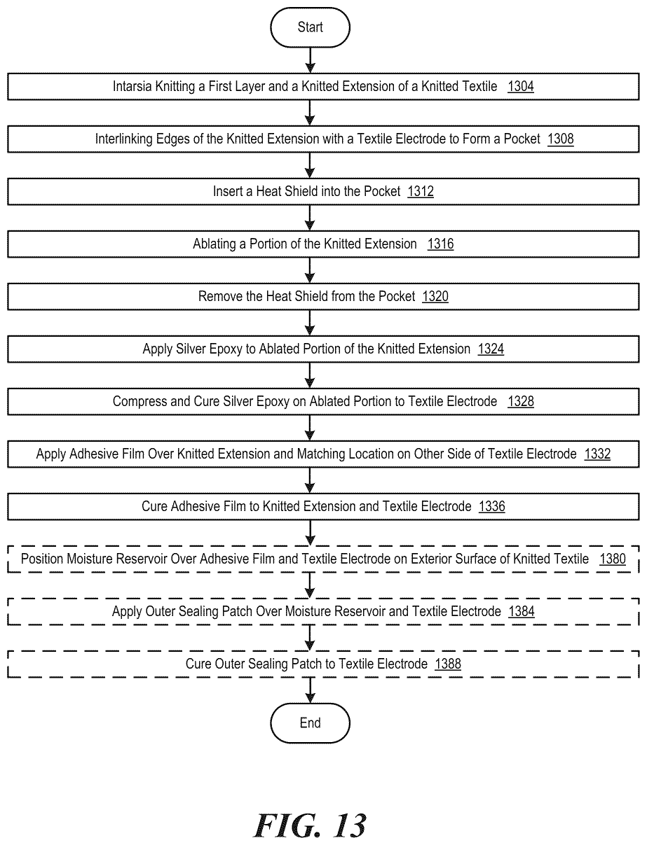

In FIG. 13, a flowchart of a process of creating an electrical connection between the textile electrode and a conductive trace of the knitted textile is shown. Steps 1380-1388 show three optional steps, denoted by dashed lines, for embodiments where a moisture reservoir is desired or required.

The embodiments of the invention described above are intended to be merely exemplary; numerous variations and modifications will be apparent to those skilled in the art. One skilled in the art will appreciate further features and advantages of the disclosure based on the above-described embodiments. Such variations and modifications are intended to be within the scope of the present invention as defined by any of the appended claims. Accordingly, the disclosure is not to be limited by what has been particularly shown and described, except as indicated by the appended claims. All publications and references cited herein are expressly incorporated herein by reference in their entirety.

What is claimed is:

1. A knitted textile, comprising:
a textile electrode region;
a conductive trace region that terminates in a knitted extension, the conductive trace region comprising one or more electrical conductors twisted with an insulator, the knitted extension configured to overlay a portion of the textile electrode region and includes an ablated area where the insulator has been removed;
conductive material, located at an intersection of the ablated area and the textile electrode region, configured to provide an electrical connection between the conductive trace region and the textile electrode region;
sealing film, placed around the conductive material, configured to protect the conductive material and seal the conductive material from one or more textile layers that surround the electrical connection; and
an outer sealing patch surrounding the textile electrode region, configured to provide a moisture barrier between the textile electrode region and the one or more surrounding textile layers.

2. The knitted textile of claim 1, wherein the textile electrode region and a portion of the conductive trace region that does not include the knitted extension are knitted in a common layer of the knitted textile.

3. The knitted textile of claim 1, wherein knit stitches at the edges of the knitted extension are interlinked with corresponding knit stitches of the textile electrode region except on one side of the knitted extension, wherein a pocket is formed between the knitted extension and the textile electrode region.

4. The knitted textile of claim 3, wherein the pocket is formed on an outside surface of the knitted textile.

5. The knitted textile of claim 1, wherein the conductive material is a silver paste compound.

6. The knitted textile of claim 1, wherein a laser creates the ablated area in the knitted extension.

7. The knitted textile of claim 1, further comprising:
a moisture reservoir, positioned above the sealing film on an outer side of the knitted textile and in contact with the textile electrode region, configured to retain moisture and enhance electrical transfer between the textile electrode region and skin of a user wearing the knitted textile; and
an outer seal patch, applied to the first side of the knitted textile and over the moisture reservoir, configured to surround the textile electrode region, limit evaporation of moisture from the moisture reservoir, and dampen electrical noise through the textile electrode region.

8. The knitted textile of claim 1, wherein insulators of the conductive trace region have a higher tensile strength than the one or more electrical conductors.

9. A knitted textile, comprising:
a textile electrode;
a conductive trace that terminates in a knitted extension, the conductive trace comprising one or more electrical conductors twisted with an insulator, the knitted extension configured to overlay a portion of the textile electrode and includes an ablated area where the insulator has been removed;
conductive epoxy, located at an intersection of the ablated area and the textile electrode, configured to provide an electrical connection between the conductive trace and the textile electrode; and
adhesive film, applied to a first side of the knitted textile around and over the ablated area and the conductive epoxy and on a second side of the knitted textile opposite the adhesive film applied to the first side, configured to provide a water-tight encapsulation of the electrical connection when cured.

10. The knitted textile of claim 9, wherein the textile electrode and a portion of the conductive trace that does not include the knitted extension are knitted in a common layer of the knitted textile.

11. The knitted textile of claim 9, wherein knit stitches at edges of the knitted extension are interlinked with corresponding knit stitches at the textile electrode except on one side of the knitted extension, wherein a pocket is formed between the knitted extension and the textile electrode.

12. The knitted textile of claim 11, wherein the pocket is formed on an outside surface of the knitted textile.

13. The knitted textile of claim 9, wherein the conductive epoxy is a silver paste compound.

14. The knitted textile of claim 9, wherein a laser creates the ablated area in the knitted extension.

15. The knitted textile of claim 9, further comprising:
a moisture reservoir, positioned above the adhesive film on an outer side of the knitted textile and in contact with the textile electrode, configured to retain moisture and enhance electrical transfer between the textile electrode and skin of a user wearing the knitted textile; and
an outer seal patch, applied to the first side of the knitted textile and over the moisture reservoir, configured to surround the textile electrode, limit evaporation of moisture from the moisture reservoir, and dampen electrical noise through the textile electrode.

16. The knitted textile of claim 9, wherein insulators of the conductive trace have a higher tensile strength than the one or more electrical conductors.

17. A method for creating an electrical connection in a knitted textile, comprising:
intarsia knitting a first layer comprising a textile electrode and a conductive trace, the conductive trace comprising one or more conducting wires twisted with an insulator, the conductive trace terminating in a knitted extension forming a second layer and overlaying a portion of the textile electrode;
ablating a portion of the knitted extension by removing the insulator from the ablated portion;
applying a silver epoxy to the ablated portion, the silver epoxy providing an electrical connection between the conductive trace and the textile electrode; and
applying an adhesive film to a first side of the knitted textile around and over the ablated area and the silver epoxy, and to a second side of the knitted textile opposite the adhesive film applied to the first side, the adhesive film providing a water-tight encapsulation of the electrical connection.

18. The method of claim 17, further comprising:
interlinking edges of the knitted extension with the textile electrode except on one side of the knitted extension, thereby forming a pocket between the knitted extension and the textile electrode.

19. The method of claim 18, further comprising:
temporarily inserting a heat shield into the pocket prior to ablating the portion of the knitted extension; and
removing the heat shield from the pocket following the ablating,
wherein the heat shield prevents damage to the knitted textile while ablating the portion of the knitted extension.

20. The method of claim 17, further comprising:
ablating the portion of the knitted extension with a laser; and
cleaning ash and debris from the ablated portion with a cleaning agent.

21. The method of claim 17, wherein in response to applying the silver epoxy to the ablated portion, the method further comprising:
covering an area surrounding the silver epoxy with a thin plastic sheet;
placing a weight over the thin plastic sheet to flatten the ablated area to the portion of the textile electrode; and
removing the weight and the thin plastic sheet after a predetermined curing time for the silver epoxy.

22. The method of claim 17, wherein in response to applying the adhesive film, the method comprises:
heating, by a heat press, the adhesive film for a first time period followed by cooling, by a cold press, the adhesive film for a second time period to cure the adhesive film.

23. The method of claim 17, further comprising:
positioning a moisture reservoir above the adhesive film on an outer side of the knitted textile and contacting the textile electrode, the moisture reservoir retaining moisture and enhancing electrical transfer between the textile electrode and skin of a user wearing the knitted textile; and
applying an outer sealing patch to the first side of the knitted textile over the moisture reservoir and surrounding the textile electrode to limit evaporation of moisture from the moisture reservoir and dampen electrical noise through the textile electrode; and
curing the outer sealing patch.

24. The method of claim 23, wherein curing the outer sealing patch comprises:
heating, by a heat press, the outer sealing patch for a first time period followed by cooling, by a cold press, the outer sealing patch for a second time period.

25. The method of claim 17, wherein the insulators of the conductive trace have a higher tensile strength than the one or more conducting wires.

26. The method of claim 17, wherein the one or more conducting wires have a non-conducting outer layer separate from the insulators of the conductive trace.

27. The method of claim 26, wherein ablating the portion of the knitted extension comprises removing the insulator from the ablated portion and the non-conducting outer layer from the one or more conducting wires.
